(19) United States
(12) Patent Application Publication
(10) Pub. No.: US 2014/0200156 A1
(43) Pub. Date: Jul. 17, 2014

(54) GENES INDUCING AGONISTIC EFFECTS BY ANTI-C-MET ANTIBODY TREATMENT AND DRUG SCREENING METHOD USING THE GENES

(71) Applicant: Samsung Electronics Co., Ltd., Suwon-si (KR)

(72) Inventors: Bo-gyou KIM, Seoul (KR); Kyung-ah KIM, Seongnam-si (KR); Dae-soon SON, Seoul (KR); Eun-jin LEE, Seongnam-si (KR)

(73) Assignee: Samsung Electronics Co., Ltd., Suwon-si (KR)

(21) Appl. No.: 14/156,212

(22) Filed: Jan. 15, 2014

(30) Foreign Application Priority Data
Jan. 15, 2013 (KR) 10-2013-0004466

(51) Int. Cl. G01N 33/50

(52) U.S. Cl. CPC G01N 33/5023
USPC 506/9; 506/16; 435/6.12

(57) ABSTRACT

Biomarkers for screening drugs reducing side effects of anti-c-Met antibodies and a method of screening using the biomarkers, and more particularly, biomarkers commonly enhancing or reducing gene expression and a method of screening anti-c-Met antibodies having reduced side effects using the biomarkers or a method of screening drugs that reduce side effects of anti-c-Met antibodies.

### CROSS-REFERENCE TO RELATED APPLICATIONS

This application claims the benefit of Korean Patent Application No. 10-2013-0004466, filed on Jan. 15, 2013 in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 14,926 Byte ASCII (Text) file named “715866_ST25.TXT,” created on Jan. 14, 2014.

### BACKGROUND

1. Field

The present disclosure relates to biomarkers for screening agonistic effects of anti-c-Met antibodies, and anti-c-Met antibodies having reduced agonistic effects using the biomarkers, or drug screening methods reducing agonistic effects of the anti-c-met antibodies. Specifically, the present disclosure relates to biomarkers commonly having enhanced or reduced gene expressions by the anti-c-met antibodies or drug screening methods that may reduce agonistic effects of the anti-c-met antibodies.

2. Description of the Related Art

Various types of genome sequencing projects have been completed and reported to the National Center for Biotechnology Information (NCBI). In order to research about gene functions based on a great amount of data obtained through these projects, a genome-wide expression research is in progress. In order to analyze the expression of thousands of genes in one experiment, DNA microarray analysis is performed.

By using recent DNA microarray technology, expression patterns of genes expressed in specific tissues after addition of chemicals (e.g., drugs) can be determined and new drug candidates may be quantitatively and qualitatively analyzed.

c-Met receptor tyrosine kinase contributes to migration, invasion and morphogenesis accompanying embryogenesis and neogenesis. However, c-Met is known for playing a certain role in tumorigenesis. The fact that reproductive cell mutants are activated in a kinase domain of c-Met is related to a development of papillary renal cell carcinoma (PRCC). Mutants in the kinase domain are rare; however, the mutants have been reported in sporadic PRCC, squamous cell carcinoma, and gastric adenocarcinoma. A simultaneous increase in the level of c-Met and HGF/SF, which is a specific ligand for the c-Met, is commonly observed in a clinically related multiple tumors. Correlations between enhanced expression, disease progression, metastasis, and mortality rate have been reported in several types of cancers, such as bladder cancer, breast cancer, and gastric adenocarcinoma, as well as in leiomyosarcoma and glioblastoma.

Accordingly, anti-c-Met antibodies for treating cancer by blocking a pathway described above have been tried in the past. However, these anti-c-Met antibodies are known to have various side effects.

Hence, there is a desire to develop a method to identify materials mitigating toxicity of the anti-c-Met antibodies (e.g., by using a microarray and the like, or by screening anti-c-Met antibodies having low toxicity).

### SUMMARY OF THE INVENTION

The invention provides a DNA microarray chip comprising one or more oligonucleotide sequences, wherein the one or more oligonucleotides is selected from a group of biomarkers as described herein.

The invention also provides a method of screening anti-c-Met antibody having reduced side effects, the method comprising: 1) treating a cancer cell with a test compound to provide an experimental group; 2) isolating RNA from the experimental group treated with the sample compound, and from a control group cancer cell that is not treated with the test compound; 3) producing cDNA from the RNA of the experimental group and the control group, and marking the cDNA of the experimental group and the control group with different fluorescent markers; 4) hybridizing the cDNA of each of the experimental and control groups to a DNA microarray chip of claim 1 to determine gene expression of the experimental and control groups; and 5) comparing the gene expression of the experimental and control group.

Also provided is a method of A method of screening identifying c-Met-antibodies having reduced side effects, the method comprising: 1) treating a cancer cell strain with a sample test compound to provide an experimental group; 2) separating isolating RNA from an the experimental group treated with the sample test compound and from a control group cancer cell that is not treated with the sample test compound; 3) processing performing a real-time reverse transcript polymerase chain reaction (RT-PCR) of with the separated RNA from the experimental group and the control group by using a primer complementary to a biomarker gene according to an embodiment and capable of amplifying the biomarker gene to produce a biomarker gene product; and 4) comparing expression of a the biomarker gene products of step 3) of the experimental group and the control group.

Additionally, the invention provides a kit for screening anti-c-Met antibodies having reduced side effects comprising the DNA microarray chip.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a process of selecting genes related to agonism;

FIG. 2 is a graph illustrating amplification and proliferation of NCI-H441 cells treated with various concentrations of anti-c-Met antibodies;

FIG. 3 is a Venn diagram of genes whose expression changed by twofold or more under two treatment conditions (antibody concentrations of 0.5 μg/mL and 1 μg/m L);

FIG. 4 is a graph that illustrates a qPCR result showing the extent of agonism of anti-c-Met antibodies in NCI-H441 cells;

FIG. 5 is a graph showing the results of a BrdU assay showing an extent of agonism of anti-c-Met antibodies in NCI-H441 cells; and

FIGS. 6A and 6B are graphs that illustrate the extent of expression of a selected gene in anti-c-Met antibody-treated Caki-1 cells.

### DETAILED DESCRIPTION OF THE INVENTION

Additional aspects will be set forth in part in the description which follows and, in part, will be apparent from the description, or may be learned by practice of the presented embodiments.

According to an aspect of the present inventive concept, there is provided genetic biomarkers related to side effects of anti-c-Met antibodies.

According to another aspect of the present inventive concept, there is provided anti-c-Met antibodies having low side effects by using genetic biomarkers or drugs reducing side effects of the anti-c-Met antibodies.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Applicants have identified biomarker genes that can be used to screen side effects of anti-c-Met antibodies. Therefore, according to an embodiment of the present inventive concept, there is provided a biomarker for screening side effects of anti-c-Met antibodies, the biomarker including genes selected from the group consisting of:

SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2 (GenBank Accession No.: NM_{—}000758.2), TNFAIP3 (GenBank Accession No.: NM_{—}006290.2), KDM6B (GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1(GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8(GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), GOS2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F (GenBank Accession No.: NM_{—}001014450.1), ITPRIP (GenBank Accession No.: NM_{—}033397.2), DCUN1 D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1 RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), SOCS3 (GenBank Accession No.: NM_{—}003955.3), BMP2 (GenBank Accession No.: M22489.1), ZNF451 (GenBank Accession No.: BC021712.2), LOC643008 (GenBank Accession No.: AK055768.1), CDKN1B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2 (GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2).

The term “c-Met”, “c-Met polypeptide”, or “c-Met receptor” as used herein refers to a tyrosine kinase receptor bonding to hepatic cell growth factor (HGF). Specific examples include a human polypeptide encoded by the nucleotide sequence of GenBank Accession No. NM_{—}000245, a human protein encoded by the nucleotide sequence of GenBank Accession No. NM_{—}000236, or an extracellular domain thereof.

The term “antibody” includes all parts having immunoglobulin-like bonding functions. The term includes a complete antibody molecule and all of antibody bonding fragments of the complete antibody molecule or a single chain, a Camelide antibody such as a nanobody, a phage-display bonding structure, and the like.

Applicants have identified the following biomarker genes as having enhanced expression after treatment with an anti-c-Met antibody:

SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2 (GenBank Accession No.: NM_{—}000758.2), TNFAIP3 (GenBank Accession No.: NM_{—}006290.2), KDM6B (GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1 (GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8 (GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), GOS2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F (GenBank Accession No.: NM_{—}001014450.1), ITPRIP (GenBank Accession No.: NM_{—}033397.2), DCUN1D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), SOCS3 (GenBank Accession No.: NM_{—}003955.3), and BMP2 (GenBank Accession No.: M22489.1).

Applicants have identified the following biomarker genes as having reduced expression after treatment with an anti-c-Met antibody:

ZNF451 (GenBank Accession No.: BC021712.2), LOC643008 (GenBank Accession No.: AK055768.1), CDKN1 B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2 (GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2).

According to another aspect of the present inventive concept, there is provided a DNA microarray chip for screening anti-c-Met antibodies having reduced side effects or for screening drugs that reduce side effects of anti-c-Met antibodies, wherein the DNA microarray chip comprises one or more oligonucleotides selected from the above-described group of biomarker genes or a portion of the oligonucleotide sequence, or a sequence complementary to the oligonucleotide sequence or portion thereof. When a portion of the biomarker sequence is used, the portion should be of sufficient length so as to specifically hybridize with the particular oligonucleotide biomarker gene target (e.g., at least to 10 consecutive nucleotides, at least 15 consecutive nucleotides, at least 20 consecutive nucleotides, at least 25 consecutive nucleotides, at least 30 consecutive nucleotides, at least 35 consecutive nucleotides, at least 40 consecutive nucleotides, at least 45 consecutive nucleotides, or at least 50 consecutive nucleotides.

In one embodiment, the DNA microarray comprises two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more) of the oligonucleotide sequences or a portion thereof or a sequence complementary to the olgionucleotide sequence or portion thereof described above. For example, the microarray can comprise 50 or more, 60 or more, 70 or more, 80 or more, 90 or more, or 100 or more different oligonucleotides of the oligonucleotide sequences described above or a portion thereof or a sequence complementary thereto.

The DNA microarray chip can comprise other oligonucleotide sequences in addition to the one or more of oligonucleotide sequences or a portion thereof or a sequence complementary to the olgionucleotide sequence or portion thereof described above. However, according to some embodiments, the DNA microarray chip has a limited number of oligonucleotides, thereby tailoring the microarray to the uses disclosed herein. For instance, in some embodiments, the DNA microarray chip can have about 1000 or fewer different oligonucleotide sequences, such as about 500 or fewer different oligonucleotide sequences, or even 100 or fewer different oligonucleotide sequences. In some embodiments, the microarray consists of one or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, 50 or more, etc.) different oligonucleotides of the group of oligonucleotides described above

The DNA microarray chip may be manufactured by a method known in the art. The method of manufacturing the microarray chip is as follows: In order to stabilize a screened biomarker on a substrate of a DNA chip as a probe DNA molecule, micropipetting using a piezoelectric method or a method of using a pin-shaped spotter is desirable; however, the method is not limited thereto and a pin-shaped spotter microarray may be used.

A substrate of the DNA microarray chip may be selected from the group consisting of amino-silane, poly-L-lysine, and aldehyde; however, the substrate is not limited thereto. Also, the substrate may be selected from the group consisting of slide glass, plastic, metal, silicon, nylon membrane, and nitrocellulose membrane; however, the substrate is not limited thereto.

According to another aspect of the present inventive concept, there is provided a method of screening for anti-c-Met antibodies having reduced side effects, the method including: 1) treating a cancer cell strain with a sample compound; 2) isolating RNA from an experimental group of cells treated with the test compound, and from a control group of cells not treated with the test compound; 3) synthesizing cDNA from the separated RNA of the experimental group and the control group, marking the cDNA from the experimental group and the control group with different fluorescent markers; 4) hybridizing the cDNA marked with different fluorescent markers to the DNA microarray chip, wherein an oligonucleotide comprising the entire or a part of the biomarker is bonded or wherein an oligonucleotide sequence complementary to the biomarker gene sequence is bonded; 5) analyzing the DNA microarray chip; and 6) comparing biomarker expression of the experimental and control groups.

The methods of screening for anti-c-Met antibodies having reduced side effects may be as follows: First, the method comprises a process of treating a cancer cell strain with a sample (i.e., test) compound (e.g., anti-c-Met antibodies and/or a sample drug). The cancer cell strain used may be a commercially usable cell strain. All directly prepared cancer cell strains and a cell strain promoted for proliferation by anti-c-Met antibodies may be used. The cancer cell strain preferably is NCI-H441 cells or Caki-1 cells.

Thereafter, the method comprises a process of separating RNA from an experimental group of cells treated with the sample drug and/or anti-c-Met antibody (i.e., the test compound) and a control group of cells that are not treated with the test compound.

Thereafter, the method comprises a process of synthesizing the separated RNA of the experimental group and the control group into cDNA, thereby marking the experimental group and the control group.

In screening, fluorescent materials may preferably be selected from the group consisting of Cy3, Cy5, poly L-lysine-fluorescein isothiocyanate (FITC), rhodamine-B-isothiocyanate (RITC), rhodamine; however, the fluorescent materials are not limited thereto and all fluorescent materials known in the art may be used.

Thereafter, the method comprises a process of hybridizing the cDNA marked with different fluorescent markers to the DNA microarray chip.

Thereafter, the method comprises a process of analyzing the DNA microarray chip.

The analysis may preferably be processed by using GenePix 4.1 software (Axon Instruments, USA), but is not limited thereto, and any software known in the art may be used.

Thereafter, the method comprises a process of comparing expression of more or more (e.g., 2, 3, 4, 5, 6, 7, 8, 9, 10, 11, 12, 13, 14, 15, 16, 17, 18, 19, 20, 30, 40, 50, 60, 70, 80, 90, 100, or more) of the above-described biomarkers in the experimental group and the control group. In one embodiment, a two-fold or more (e.g., three-fold, four-fold, five-fold, six-fold, seven-fold, eight-fold, nine-fold, ten-fold or more) difference in expression of one or more biomarker genes between the experimental and control groups identifies a drug that reduces side effects of an anti-c-Met antibody or an anti-c-Met antibody with reduced side effects relative to a control anti-c-Met antibody (e.g., 5D5 antibody).

In another aspect, the method of screening for anti-c-Met antibodies having reduced side effects may comprise: (1) obtaining a sample of cancer cells, (2) dividing the sample into an experimental group and a control group, wherein the experimental group is treated with a test anti-c-Met antibody and the control group is treated with a control anti-C-Met antibody, (3) obtaining RNA from the experimental group and the control group; (4) producing cDNA from the RNA of the experimental group and the control group, wherein the cDNA comprises one or more fluorescent markers; (5) hybridizing the cDNA to the DNA microarray chip comprising the biomarker genes; and (6) comparing the expression of the one or more oligonucleotide sequences or a portion thereof or a sequence complementary to the oligonucleotide sequence or portion thereof in the experimental group and the corresponding expression in the control group, wherein a two-fold or more difference in expression between the experimental and control groups identifies an anti-c-Met antibody having reduced side effects.

According to another aspect of the present inventive concept, there is provided a method of screening a drug that reduces side effects of anti-c-Met antibodies, the method including 1) treating a cancer cell strain with a test compound; 2) isolating RNA from an experimental group cell treated with the test compound and a control group cell that is not treated with the sample compound; 3) synthesizing cDNA from the isolated RNA of the experimental group and the control group, and marking the cDNA of the experimental group and the control group with different fluorescent markers; 4) hybridizing the cDNA marked with different fluorescent markers to the DNA microarray chip, wherein an oligonucleotide comprising the entire or a part of the biomarker is bonded or wherein an oligonucleotide sequence complementary to the biomarker gene sequence is bonded; 5) analyzing the DNA microarray chip; and 6) comparing a biomarker expression from data of 5) and the control group.

According to another aspect, the method of screening for drug that reduces side effects of anti-c-Met antibodies may comprise (1) obtaining a sample of cancer cells, (2) dividing the sample into an experimental group and a control group that are treated with an anti-c-Met antibody, wherein the experimental group is also treated with a test compound, (3) obtaining RNA from the experimental group and the control group; (4) producing cDNA from the RNA of the experimental group and the control group, wherein the cDNA comprises one or more fluorescent markers; (5) hybridizing the cDNA to the DNA microarray chip; and (6) comparing the expression of the one or more oligonucleotide sequences or a portion thereof or a sequence complementary to the oligonucleotide sequence or portion thereof in the experimental group and the corresponding expression in the control group, wherein a two-fold or more difference in expression between the experimental and control groups identifies a drug that reduces side effects of an anti-c-Met antibody.

Any cancer cell strain may be used as the cancer cell strain and a cell strain promoting proliferation may be used. Also, the cancer cell strain may preferably be NCI-H441 cells or Caki-1 cells.

All aspects of the method are otherwise as described with respect to other methods and compositions discussed herein.

According to another aspect of the present inventive concept, there is provided a method of screening c-Met-antibodies having reduced side effects, the method including:1) treating a cancer cell strain with a test compound; 2) isolating RNA from an experimental group cell treated with the test compound and a control group cell that is not treated with the test compound; 3) processing a real-time reverse transcript polymerase chain reaction (RT-PCR) of the isolated RNA by using a primer complementary to a biomarker gene and capable of amplifying the biomarker gene; and 4) comparing expression of a gene product of step 3) to a control group.

The method of screening may be as follows: First, a cancer cell strain is treated with a test compound. The cancer cell strain used may be a commercially usable cell strain. Any of directly prepared cancer cell strains and a cell strain promoted for a proliferation by anti-c-Met antibodies may be used. Also, the cancer cell strain may preferably be NCI-H441 cell or Caki-1 cell.

Thereafter, there is provided a process of isolating RNA from an experimental group treated with the test compound and a control group that is not treated with the test compound.

Thereafter, real-time reverse transcript polymerase chain reaction (RT-PCR) is performed on the RNA using a primer complementary to a biomarker gene according to an embodiment and capable of amplifying the biomarker gene.

The primer may be about 16 mer to about 35 mer polynucleotide specifically amplifying the biomarker gene according to an embodiment of the present inventive concept. The primer may be about 18 mer to about 25 mer, and may specifically amplify the biomarker according to an embodiment of the present inventive concept, and a location of the primer is not limited. Also, the primer may be a forward-direction and a reverse-direction primer selected from the group consisting of SEQ ID NOs: 1 to 40.

Thereafter, expression of a gene product of the experimental group is compared to a control group.

In another aspect, the method of screening for an anti-c-Met antibody with reduced side effects comprises: (1) obtaining a sample of cancer cells, (2) dividing the sample into an experimental group and a control group, wherein the experimental group is treated with a test anti-c-Met antibody and the control group is treated with a control anti-C-Met antibody, (3) obtaining RNA from the experimental group and the control group; (4) performing a real-time reverse transcript polymerase chain reaction (RT-PCR) with the RNA from the experimental group and the control group using a primer complementary to a biomarker gene and capable of amplifying the biomarker gene to produce a biomarker gene product; and (5) comparing expression of the biomarker gene product of step (4) in the experimental group and the control group, wherein a two-fold or more difference in expression between the experimental and control groups identifies an anti-c-Met antibody having reduced side effects, and wherein the biomarker gene is selected from a particular group (i.e., the group of biomarker genes described above relative to the DNA microarray chip).

According to another aspect of the present inventive concept, there is provided a method of screening a drug that reduces side effects of anti-c-Met antibodies, the method including: 1) treating a cancer cell strain with a test compound; 2) isolating RNA from an experimental group treated with the test compound and a control group that is not treated with the test compound; 3) performing real-time reverse transcript polymerase chain reaction (RT-PCR) of the isolated RNA using a primer complementary to a biomarker gene described above and capable of amplifying the biomarker gene; and 4) comparing expression of the gene product of the experimental group to a control group.

The primer may be about 16 mer to about 35 mer polynucleotides specifically amplifying the biomarker gene according to an embodiment of the present inventive concept. The primer may be about 18 mer to about 25 mer, and may specifically amplify the biomarker according to an embodiment of the present inventive concept, and a location of the primer is not limited. Also, the primer may be a forward-direction and a reverse-direction primer selected from the group consisting of sequence numbers 1 to 40.

According to another aspect of the present inventive concept, there is provided a method of identifying drug that reduces side effects of an anti-c-Met antibody. The method comprises: (1) obtaining a sample of cancer cells, (2) dividing the sample into an experimental group and a control group that are treated with an anti-c-Met antibody, wherein the experimental group is also treated with a test compound, (3) obtaining RNA from the experimental group and the control group; (4) performing a real-time reverse transcript polymerase chain reaction (RT-PCR) with the RNA from the experimental group and the control group using a primer complementary to a biomarker gene and capable of amplifying the biomarker gene to produce a biomarker gene product; and (5) comparing expression of the biomarker gene product of step (4) in the experimental group and the control group, wherein a two-fold or more difference in expression between the experimental and control groups identifies a drug that reduces side effects of an anti-c-Met antibody, and wherein the biomarker gene is selected from a particular group (i.e., the group of biomarker genes described above relative to the DNA microarray chip).

According to another aspect of the present inventive concept, there is provided a kit for screening anti-c-Met antibodies having reduced side effects including a microarray according to an embodiment or a kit for screening drugs that reduce side effects of the anti-c-Met antibodies.

In a screening kit, a fluorescent material may further be added, and the fluorescent material may be selected from the group consisting of strepavidin-like phosphatase conjugate, chemifluorescence, and chemiluminescent. In some embodiments, Cy3 and Cy5 may be used.

Also, a reaction reagent may be additionally included in the screening kit, and the reaction reagent may include a buffer solution for a hybridization, a reverse transcriptase for synthesizing cDNA from RNA, cNTPs and rNTP (pre-mixed or separately supplied), a labeling reagent such as a chemical inducer of a fluorescent dye, or washing buffer solution and the like, but is not limited thereto and all reagents needed in a hybridization reaction of a DNA microarray chip known in the art may be included.

According to another aspect of the present inventive concept, there is provided a kit for screening anti-c-Met antibodies having reduced side effects or a kit for screening a drug that reduces side effects of anti-c-Met antibodies including a primer set of an embodiment.

The primer may be at least two forward-direction or reverse-direction primers selected from the group consisting of SEQ ID NOs: of 1 to 40. Preferably, the primer may be any one sequence selected from the group consisting of SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, and SEQ ID NOs: 39 and 40.

By using the kit and the methods of embodiments of the present inventive concept, side effects of anti-c-Met antibodies may be efficiently detected. By using the methods, anti-c-Met antibodies having reduced side effects may be rapidly and precisely screened. As a result, a speed of developing a new antibody medicine may be increased. Also, by finding a gene related to agonism of anti-c-Met antibodies, candidates having greater antitumor effects may be obtained than when treated with the anti-c-Met antibodies. Also, through a method of securing a gene family by using the microarray, a rapid screening of a drug having reduced side effects as well as finding a material related to a drug resistance are possible. Furthermore, through understanding a mechanism of side effects of a drug, the reactivity of a drug may be predicted and as a result, a diagnostic marker may be developed.

### EXAMPLES

It should be understood that the exemplary embodiments described herein should be considered in a descriptive sense only and not for purposes of limitation. Descriptions of features or aspects within each embodiment should typically be considered as available for other similar features or aspects in other embodiments.

### Example 1

### Gene Contributing to Side Effects of Anti-c-Met Antibodies

1-1. Verifying Side Effects of Anti-c-Met Antibodies

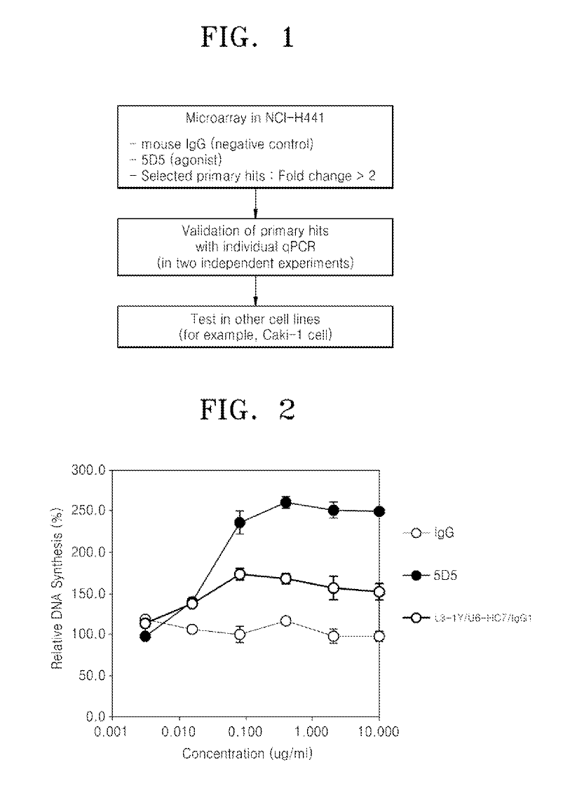

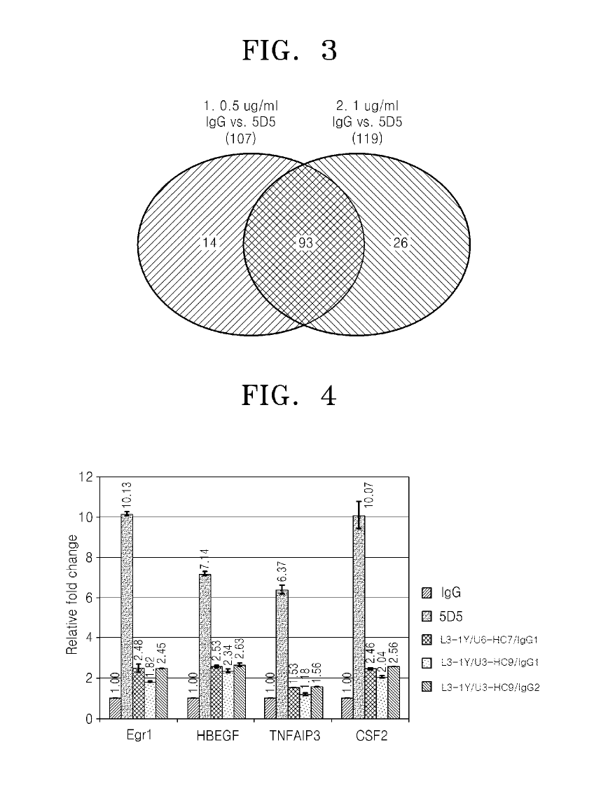

Side effects of anti-c-Met antibodies are known. To investigate how to prevent the side effects of the anti-c-Met antibodies, a BrdU assay was performed by using an NCI-H441 cell strain that is a non-small cell lung cancer cell line. The BrdU assay is an assay verifying the extent of DNA synthesis through dying BrdU, and mouse IgG was used as a control group. A well-known agonist, 5D5 antibody (used after purifying from the hybridoma from ATCC, cat. no. HB-11895) was verified to promote proliferation. In the case of L3-1Y TH7 (independently prepared, SEQ ID NO: 41), a lower agonism compared to that of 5D5 was shown (FIG. 2).

1-2. Processing Microarray after Treating with Anti-c-Met Antibodies

To find a gene reflecting side effects of anti-c-Met antibodies, a microarray was processed after treating with a well-known agent 5D5. First, to extract RNA for the microarray, NCI-H441 cells (ATCC) were divided into 6 well plates, each having a cell concentration of 4.5×10⁵ cells and cultivated for 2 days. Mouse IgG used as a negative control group and 5D5 were diluted in 2 different concentrations, 0.5 μg/mL and 1 μg/mL in RPMI1640 (GIBCO) without FBS (fetal bovine serum) and then treated for 2 hours. After treating for 2 hours, RNA was extracted using RNeasy Mini kit (Qiagen, #74106). For performing a microarray, RNA was prepared in 3 repeated samples with respect to an IgG treatment group and a 5D5 treatment group. The HG-U219 chip from Affimatrix was used for the microarray and DNA LINK company performed a microarray analysis.

1-3. Selecting Gene Related to Agonism of Anti-c-Met Antibodies

For collecting images, Affymetrix GeneChip Scanner 3000 7G was used and data was extracted by using Affymetrix Command Console software. The extracted data was processed through a robust multi-array average (RMA) normalization to find differentially expressed genes (DEG). The genes processed through the normalization were statistically treated by using unpaired T-Test (P-value <0.05). Genes having a difference of twofold or more compared to an average were selected.

As shown in Table 1, when the genes were treated at a concentration of 0.5 μg/mL, a total of 107 genes had a difference of twofold or more. 96 genes showed enhanced expression, whereas 11 genes showed reduced expression.

When treated at a concentration of 1 μg/mL, 109 genes showed enhanced expression. 10 genes showed reduced expression such that a total of 119 genes showed a difference of twofold or more. 93 genes were common between genes changing when treated at a concentration of 0.5 μg/mL and genes changing when treated at a concentration of 1 μg/mL. A total of 133 genes (FIG. 3) show a difference of twofold or more at a concentration of at least 0.5 μg/mL.

Table 2 shows fold change values of genes having enhanced expression when treated with 5D5, and Table 3 shows fold change values of genes having reduced expression when treated with 5D5.

TABLE 1
Number of genes having a difference of twofold or more in each treatment
group of a microarray

| | Enhanced | Reduced | |
|---|---|---|---|
| Index | expression | expression | Total genes |
| IgG vs SD5 (0.5 μg/mL) | 96 | 11 | 107 |
| IgG vs SD5 (1 μg/mL) | 109 | 10 | 119 |
| Total genes | 119 | 14 | 133 |

TABLE 2
Genes having enhanced expression when treated with 5D5 in a microarray.

| | | | Fold change | |
|---|---|---|---|---|
| Representative | | | (cDNA microarray) | |
| Public ID | Gene Title | Gene Symbol | 0.5 ug/ml | 1 ug/ml |
| AK226147.1 | sprouty homolog 4 | SPRY4 | 8.9006 | 7.9156 |
| | (Drosophila) | | | |
| BC073983.1 | early growth response 1 | EGR1 | 8.2632 | 7.3552 |
| BT007067.1 | interleukin 8 | IL8 | 7.3479 | 8.1883 |
| AK222688.1 | haparin-binding EGF-like | HBEGF | 5.9960 | 5.8956 |
| | growth factor | | | |
| NM_001136178.1 | early growth response 2 | EGR2 | 5.8874 | 5.9993 |
| CX756248 | ras homolog gene family, | RHOB | 5.2341 | 4.9930 |
| | member B | | | |
| NM_016270.2 | Kruppel-like factor 2 | KLF2 | 4.5512 | 4.2099 |
| | (lung) | | | |
| CR607047.1 | polo-like kinase 3 | PLK3 | 4.2826 | 4.0284 |
| NM_006945.4 | small proline-rich | SPRR2D | 4.2557 | 4.2976 |
| | protein 2D | | | |
| NM_000758.2 | colony stimulating factor | CSF2 | 4.1381 | 4.1675 |
| | 2 (granulocyte- | | | |
| | macrophage) | | | |
| NM_006290.2 | tumor necrosis factor, | TNFAIP3 | 4.0601 | 3.9999 |
| | alpha-induced protein 3 | | | |
| BU685696 | lysine (K)-specific | KDM6B | 3.7481 | 4.0303 |
| | demethylase 6B | | | |
| NM_004864.2 | growth differentiation | GDF15 | 3.6840 | 3.2872 |
| | factor 15 | | | |
| BC005047.1 | dual specificity | DUSP6 | 3.6760 | 3.5200 |
| | phosphatase 6 | | | |
| NM_001024209.2 | small proline-rich | SPRR2E | 3.5875 | 4.3045 |
| | protein 2E | | | |
| NM_004419.3 | dual specificity | DUSP5 | 3.4749 | 3.7039 |
| | phosphatase 5 | | | |
| AI446767 | chemokine (C-—X—C motif) | CXCL2 | 3.4379 | 3.3432 |
| | ligand 2 | | | |
| AK298659.1 | FBJ murine osteosarcoma | FOS | 3.3828 | 3.1288 |
| | viral oncogene homolog | | | |
| NM_004561.2 | ovo-like 1 (Drosophila) | OVOL1 | 3.2345 | 2.9883 |
| NM_012323.2 | v-maf musculoaponeurotic | MAFF | 3.2325 | 2.8530 |
| | fibrosarcoma oncogene | | | |
| | homolog F (avian) | | | |
| NM_013246.2 | cardiotrophin-like | CLCF1 | 3.1525 | 2.9841 |
| | cytokine factor 1 | | | |
| BM904784 | zinc finger protein 697 | ZNF697 | 3.1298 | 3.6728 |
| NM_013376.3 | SERTA domain containing 1 | SERTAD1 | 3.1233 | 3.1611 |
| AI570795 | leukemia inhibitory factor | LIF | 3.0881 | 2.9576 |
| | (cholinergic | | | |
| | differentiation factor) | | | |
| NM_001901.2 | connective tissue growth | CTGF | 3.0409 | 3.3680 |
| | factor | | | |
| NM_001965.2 | early growth response 4 | EGR4 | 2.9736 | 2.5362 |
| NM_203411.1 | transmembrane protein 88 | TMEM88 | 2.8766 | 3.6745 |
| BM129310 | cysteine-serine-rich | CSRNP1 | 2.8568 | 2.9518 |
| | nuclear protein 1 | | | |
| BC018340.1 | zinc finger protein 36, | ZFP36L1 | 2.7507 | 2.7158 |
| | C3H type-like 1 | | | |
| NM_004431.2 | EPH receptor A2 | EPHA2 | 2.7311 | 2.7475 |
| NM_000958.2 | prostaglandin E receptor 4 | PTGER4 | 2.7257 | 2.7513 |
| | (subtype EP4) | | | |
| NM_006732.2 | FBJ murine osteosarcoma | FOSB | 2.6927 | 3.6858 |
| | viral oncogene homolog B | | | |
| AK298716.1 | AT rich interactive domain | ARID3B | 2.6919 | 2.7183 |
| | 3B (BRIGHT-like) | | | |
| NM_139314.1 | angiopoietin-like 4 | ANGPTL4 | 2.6628 | 3.0131 |
| CB991991 | pleckstrin homology-like | PHLDA2 | 2.6278 | 2.4896 |
| | domain, family A, member 2 | | | |
| NM_001001870.1 | chromosome 17 open reading | C17orf91 | 2.6151 | 2.1248 |
| | frame 91 | | | |
| NM_001114735.1 | BCL2-related protein A1 | BCL2A1 | 2.6026 | 2.7741 |
| AK295430.1 | cysteine-rich, angiogenic | CYR61 | 2.5870 | 2.6232 |
| | inducer, 61 | | | |
| BC063625.1 | keratin associated protein | LOC730755 | 2.5822 | 2.6457 |
| | 2-4-like | | | |
| AF370364.1 | major facilitator | MFSD2A | 2.5819 | 2.4843 |
| | superfamily domain | | | |
| | containing 2A | | | |
| NM_002135.3 | nuclear receptor subfamily | NR4A1 | 2.5677 | 2.6778 |
| | 4, group A, member 1 | | | |
| AY029180.1 | plasminogen activator, | PLAUR | 2.5836 | 2.7159 |
| | urokinase, receptor | | | |
| g197383031 | keratin 17 | KRT17 | 2.5585 | 2.5880 |
| NM_078467.1 | cyclin-dependent kinase | CDKN1A | 2.5384 | 2.5702 |
| | inhibitor 1A (p21, Cip1) | | | |
| NM_002090.2 | chemokine (C-—X—C motif) | CXCL3 | 2.5211 | 2.4604 |
| | ligand 3 | | | |
| AF123760.1 | ceroid-lipofuscinosis, | CLN8 | 2.4985 | 2.0606 |
| | neuronal 8 (epilepsy, | | | |
| | progressive with mental | | | |
| | retardation) | | | |
| NM_003125.2 | small proline-rich protein | SPRR1B | 2.4834 | 2.7396 |
| | 1B | | | |
| BF589790 | adrenomedullin | ADM | 2.4703 | 2.6062 |
| NM_002229.2 | jun B proto-oncogene | JUNB | 2.4170 | 2.4186 |
| AK301679.1 | keratin 16 /// keratin 16 | KRT16 /// | 2.4138 | 2.3152 |
| | pseudogene 1 /// keratin 16 | KRT16P1 /// | | |
| | pseudogene 2 /// keratin 16 | KRT16P2 /// | | |
| | pseudogene 3 | KRT16P3 | | |
| NM_005261.2 | GTP binding protein | GEM | 2.4061 | 2.7296 |
| | overexpressed in skeletal | | | |
| | muscle | | | |
| BC082238.1 | basic helix-loop-helix | BHLHE40 | 2.3728 | 2.2310 |
| | family, member e40 | | | |
| D78579.1 | nuclear receptor subfamily | NR4A3 | 2.3500 | 2.7104 |
| | 4, group A, member 3 | | | |
| AK294197.1 | — | ACSL4 | 2.3475 | 2.5305 |
| NM_025194.2 | inositol 1,4,5- | ITPKC | 2.3452 | 2.5210 |
| | triphosphate 3-kinase C | | | |
| AK022624.1 | apoptosis enhancing | AEN | 2.2741 | 2.0720 |
| | nuclease | | | |
| BC024145.2 | TNF receptor-associated | TRAF1 | 2.2709 | 2.7560 |
| | factor 1 | | | |
| AB044805.1 | 6-phosphofructo-2- | PFKFB2 | 2.2303 | 2.1627 |
| | kinase/fructose-2,6- | | | |
| | biphosphatase 2 | | | |
| NM_207330.1 | NIPA-like domain containing 1 | NIPAL1 | 2.2265 | 1.9434 |
| NM_025195.2 | tribbles homolog 1 | TRIB1 | 2.2228 | 2.4353 |
| | (Drosophila) | | | |
| NM_032505.2 | kelch repeat and BTB (POZ) | KBTBD8 | 2.2180 | 2.3913 |
| | domain containing 8 | | | |
| BX097024 | death inducer-obliterator 1 | DIDO1 | 2.2101 | 2.1894 |
| NM_003461.4 | zyxin | ZYX | 2.2019 | 2.2124 |
| AK301679.1 | keratin 16 pseudogene 1 | KRT16P1 | 2.2001 | 2.2216 |
| BC110820.1 | pleckstrin homology-like | PHLDA1 | 2.1848 | 2.3028 |
| | domain, family A, member 1 | | | |
| NM_001002914.2 | potassium channel | KCTD11 | 2.1784 | 2.1604 |
| | tetramerisation damain | | | |
| | containing 11 | | | |
| NM_003897.3 | immediate early response 3 | IER3 | 2.1759 | 2.0806 |
| BE874862 | GO/G1switch 2 | GOS2 | 2.1618 | 2.0444 |
| NM_005438.3 | FOS-like antigen 1 | FOSL1 | 2.1576 | 2.0806 |
| AK300470.1 | CD274 molecule | CD274 | 2.1464 | 2.1092 |
| NM_004418.3 | dual specificity | DUSP2 | 2.1424 | 2.0834 |
| | phosphatase 2 | | | |
| BC107735.1 | myeloid cell leukemia | MCL1 | 2.1355 | 2.5063 |
| | sequence 1 (BCL2-related) | | | |
| NM_015193.3 | activity-regulated | ARC | 2.1279 | 2.2700 |
| | cytoskeleton-associated | | | |
| | protein | | | |
| NM_001040619.1 | activating transcription | ATF3 | 2.1207 | 2.5239 |
| | factor 3 | | | |
| BG491844 | jun proto-oncogene | JUN | 2.1160 | 2.0518 |
| NM_002405.2 | MFNG O-fucosylpeptide 3- | MFNG | 2.0949 | 1.3421 |
| | beta-N- | | | |
| | acetylglucosaminyltransferase | | | |
| BC023512.2 | CCR4 carbon catabolite | CCRN4L | 2.0894 | 2.2203 |
| | repression 4-like (S. cerevisiae) | | | |
| BC136404.1 | epiregulin | EREG | 2.0856 | 2.1613 |
| NM_001511.1 | chemokine (C-—X—C motif) | CXCL1 | 2.0754 | 2.7372 |
| | ligand 1 (melanoma growth | | | |
| | stimulating activity, alpha | | | |
| AB065434.1 | TP53 regulating kinase | TP53RK | 2.0750 | 1.9811 |
| BC118558.1 | meteorin, glial cell | METRNL | 2.0725 | 2.0179 |
| | differentiation regulator- | | | |
| | like | | | |
| BC101639.1 | SERTA domain containing 2 | SERTAD2 | 2.0681 | 1.7855 |
| BC013906.2 | hypothetical protein | FLJ36031 | 2.0654 | 2.0208 |
| | FLJ36031 | | | |
| BC039733.1 | pentraxin 3, long | PTX3 | 2.0583 | 2.0753 |
| AF509504.1 | DOT1-like, histone H3 | DOT1L | 2.0575 | 2.0790 |
| | methyltransferase (S. cerevisiae) | | | |
| NM_004591.2 | chemokine (C-C motif) | CCL20 | 2.0561 | 2.2216 |
| | ligand 20 | | | |
| AF290426.1 | CD83 molecule | CD83 | 2.0547 | 2.0720 |
| CN364627 | angiomotin like 2 | AMOTL2 | 2.0501 | 1.9624 |
| CR602714.1 | jumonji domain containing 6 | JMJD6 | 2.0469 | 1.9682 |
| BC036731.1 | zinc finger and BTB domain | ZBTB24 | 2.0419 | 2.1278 |
| | containing 24 | | | |
| BC030702.1 | microcephalin 1 | MCPH1 | 2.0223 | 1.9821 |
| AK302213.1 | heterogeneous nuclear | HNRNPC | 2.0140 | 1.5853 |
| | ribonucleoprotein C (C1/C2) | | | |
| BQ003857 | nuclear receptor | NCOA7 | 2.0130 | 2.3423 |
| | coactivator 7 | | | |
| AF24815.1 | suppressor of cytokine | SOCS4 | 2.0108 | 1.6444 |
| | signaling 4 | | | |
| AK299391.1 | dual specificity | DUSP1 | 2.0067 | 2.3862 |
| | phosphatase 1 | | | |
| AF098290.1 | CDC42 effector protein (Rho | CDC42EP2 | 2.0007 | 1.8288 |
| | GTPase binding) 2 | | | |
| NM_024640.3 | yrdC domain containing (E. coli) | YRDC | 1.9921 | 2.0648 |
| BX356243 | protease, serine, 22 | PRSS22 | 1.9867 | 2.0080 |
| NM_138395.2 | methionyl-tRNA | MARS2 | 1.9644 | 2.1167 |
| | synthetase 2, mitochondrial | | | |
| NM_005988.2 | small proline-rich protein | SPRR2A | 1.9472 | 2.1754 |
| | 2A | | | |
| NM_052901.2 | solute carrier family 25 | SLC25A25 | 1.9187 | 2.0810 |
| | (mitochondrial carrier, | | | |
| | phosphate carrier), member 25 | | | |
| BC013734.1 | prostagladin-endoperoxide | PTGS2 | 1.9016 | 2.5466 |
| | synthase 2 (prostaglandin G/H | | | |
| | synthase and cyclooxygenase) | | | |
| NM_182919.2 | toll-like receptor adaptor | TICAM1 | 1.8912 | 2.0907 |
| | molecule 1 | | | |
| BC042362.1 | ribosomal protein S37a | RPS7A | 1.8903 | 2.0472 |
| NM_001014450.1 | small proline-rich protein 2F | SPRR2F | 1.8855 | 2.1006 |
| NM_033397.2 | inositol 1,4,5-triphosphate | ITPRIP | 1.8817 | 2.0102 |
| | receptor interacting protein | | | |
| NM_173475.2 | DCN1, defective in cullin | DCUN1D3 | 1.8769 | 2.0467 |
| | meddylation 1, domain containing | | | |
| | 3 (S. cerevisiae) | | | |
| NM_144569.4 | SPOC domain containing 1 | SPOCD1 | 1.8707 | 2.0651 |
| AF429970.1 | sterile alpha motif domain | SAMD4A | 1.8624 | 2.3503 |
| | containing 4A | | | |
| NM_001128850.1 | Ras-related associated with | RRAD | 1.8609 | 2.0430 |
| | diabetes | | | |
| NM_001145652.1 | chromosome 6 open reading frame | C6orf141 | 1.8429 | 2.2470 |
| | 141 | | | |
| AK296851.1 | laminin, beta 3 | LAMB3 | 1.8374 | 2.1213 |
| AK303389.1 | interleukin 1 receptor-like 1 | IL1RL1 | 1.8289 | 3.2112 |
| AK293280.1 | semaphorin 7A, GPI membrane | SEMA7A | 1.8156 | 2.3270 |
| | anchor (John Milton Hagen blood | | | |
| | group) | | | |
| AK304370.1 | zinc finger protein 562 | ZNF562 | 1.7945 | 2.0668 |
| AA702805 | chromosome 8 open reading frame 4 | C8orf4 | 1.7828 | 2.2224 |
| NM_003954.2 | mitogen-activated protein kinase | MAP3K14 | 1.7810 | 2.2046 |
| | kinase kinase 14 | | | |
| NM_003955.3 | suppressor of cytokine signaling 3 | SOCS3 | 1.7565 | 2.1084 |
| M22489.1 | bone morphogenetic protein 2 | BMP2 | 1.5033 | 2.2649 |

TABLE 3
Genes having reduced expression when treated with 5D5 in a microarray.

| | | | Fold change | |
|---|---|---|---|---|
| Representative | | | (cDNA microarray) | |
| Public ID | Gene Title | Gene Symbol | 0.5 ug/ml | 1 ug/ml |
| BC021712.2 | zinc finger protein 451 | ZNF451 | 0.5777 | 0.4995 |
| AK055768.1 | hypothetical protein | LOC643008 | 0.5169 | 0.4851 |
| | LOC643008 | | | |
| NM_004064.3 | cyclin-dependent kinase | CDKN1B | 0.5147 | 0.4902 |
| | inhibitor 1B (p27, Nip1) | | | |
| NM_001632.3 | alkaline phosphatase, | ALPP | 0.4994 | 0.5069 |
| | placental | | | |
| NM_198586.2 | NHI, repeat containing 1 | NHLRC1 | 0.4913 | 0.3974 |
| NM_020787.2 | zinc finger protein 624 | ZNF624 | 0.4799 | 0.5282 |
| AU147415 | FERM domain containing 4B | FRMD4B | 0.4747 | 0.6316 |
| AK299614.1 | kelch repeat and BTB (POZ) | KBTBD7 | 0.4499 | 0.5470 |
| | domain containing 7 | | | |
| CR622974.1 | hypothetical LOC100506379 | LOC100506379 | 0.3626 | 0.4015 |
| NM_001144869.1 | lipoyl (octanoyl) transferase | LIPT2 | 0.3475 | 0.4259 |
| | 2 (putative) | | | |
| NM_033503.3 | Bcl2 modifying factor | BMF | 0.3457 | 0.3166 |
| NM_024508.3 | zinc finger, BED-type | ZBED2 | 0.3379 | 0.3507 |
| | containing 2 | | | |
| NM_001198.3 | PR domain containing 1, with | PRDM1 | 0.2517 | 0.2093 |
| | ZNF domain | | | |
| NM_003106.2 | SRY (sex determining region | SOX2 | 0.2072 | 0.2105 |
| | Y)-box 2 | | | |

### Example 2

### Verification Through qPCR of Selected Genes

To verify genes selected by a microarray, qPCR was performed for each gene. Genes for verification primarily include genes having a great change in expression in two different concentrations such as genes having a notable P-value in three repeated samples and genes related to cell proliferation that is deeply related to side effects. Table 4 is a PCR primer sequence used in a verification process.

The qPCR for verification includes cell culture, RNA extraction, cDNA synthesis, and qPCR reaction. First, to extract RNA, NCI-H441 cells (ATCC) were divided into 6 well plates, each having a cell concentration of 4.5×10⁵ cells and cultured for two days. A mouse IgG used as a negative control group and 5D5 were diluted in two different concentrations of 0.5 μg/mL and 1 μg/mL in RPMI1640 (GIBCO) without FBS and treated for two hours. After treating for two hours, RNA was extracted by using RNeasy Mini kit (Qiagen, #74106). When extracting the RNA, 40 μL of RNase-free DW was used. 12 μL of the RNA was synthesized into cDNA by using a Transcriptor First Strand cDNA synthesis kit (Roche, #04 896 866 001). The cDNA was synthesized by following a protocol of the manufacturer. A qPCR reaction uses LC480 SYBR Green I Master (Roche, #04 887 352 001) and LightCycler 480 Real-Time PCR System (Roche). As an internal control group for correcting an amount of RNA sample, HPRT1 was used and qPCR was used by the following process with respect to all primers. Step 1: 95 r, 10 min; Step 2 (45 cycles): Step 2-1: 95° C., 10 sec; Step 2-2: 60° C., 20 sec; Step 2-3: 72° C., 20 sec; Step 3: 95° C., 5 sec; Step 4: 65° C., 1 min; Step 5: 95° C., continuous (every 5° C.); Step 6: 40° C., 10 sec.

Table 5 shows verification of a result of a microarray through qPCR. 17 genes having enhanced expression by 5D5 and 2 genes having reduced expression were tested and the result thereof supported the microarray result as shown in Table 5.

TABLE 4
A primer sequence used in a verification process using qPCR.

| Representative | | Gene | PCR primer sequence (5′ -> 3′) | |
|---|---|---|---|---|
| Public ID | Gene Title | Symbol | sense | antisense |
| AK226147.1 | sprouty homolog 4 | SPRY4 | ccccggcttcaggattta | ctgcaaaccgctcaatacag |
| | (Drosophila) | | (Sequence No. 1) | (Sequence No. 2) |
| BC073983.1 | early growth | EGR1 | tcggacatgacagcaacct | tttcccctttccctttagca |
| | response 1 | | (Sequence No. 3) | (Sequence No. 4) |
| BT007067.1 | interleukin 8 | IL8 | agacagcagagcacacaagc | atggttccttccggtggt |
| | | | (Sequence No. 5) | (Sequence No. 6) |
| AK222688.1 | Heparin-binding EGF- | HBEGF | tggggcttctcatgtttagg | catgcccaacttcactttctc |
| | like growth factor | | (Sequence No. 7) | (Sequence No. 8) |
| NM_001186178.1 | early growth | EGR2 | ttgaccagatgaacggagtg | tggtttctaggtgcagagacg |
| | response 2 | | (Sequence No. 9) | (Sequence No. 10) |
| CX756248 | ras homolog gene | RHOB | tatgtggccgacattgagg | gcggtcgtagtcctcctg |
| | family, member B | | (Seuence No. 11) | (Sequence No. 12) |
| NM_016270.2 | Kruppel-like | KLF2 | catctgaaggcgcatctg | cgtgtgctttcggtagtgg |
| | factor 2 (lung) | | (Sequence No. 13) | (Sequence No. 14) |
| NM_000758.2 | colony stimulating | CSF2 | tctcagaaatgtttgacctcca | gcccttgagcttggtgag |
| | factor 2 (granulocyte- | | (Sequence No. 15) | (Sequence No. 16) |
| | macrophage) | | | |
| NM_006290.2 | tumor necrosis factor, | TNFAIP3 | tgcacactgtgtttcatcgag | acgctgtgggactgactttc |
| | alpha-induced protein 3 | | (Sequence No. 17) | (Sequence No. 18) |
| BC005047.1 | dual specificity | DUSP6 | cgactggaacgagaatacgg | ggagaactcggcttggaact |
| | phosphatase 6 | | (Sequence No. 19) | (Sequence No. 20) |
| AI446767 | chemokine (C-X-C motif) | CXCL2 | cccatggttaagaaaatcatcg | cttcaggaacagccaccaat |
| | ligand 2 | | (Sequence No. 21) | (Sequence No. 22) |
| NM_013246.2 | cardiotrophin-like | CLCF1 | ccagaaaacctatgacctcacc | gggcccaggtagttcagata |
| | cytokine factor 1 | | (Sequence No. 23) | (Sequence No. 24) |
| NM_013376.3 | SERTA domain | SERTAD1 | tctgattggccgctagtga | cgactgccagaggttcctt |
| | containing 1 | | (Sequence No. 25) | (Sequence No. 26) |
| NM_001901.2 | connective tissue | CTGF | ctcctgcaggctagagaagc | gatgcactttttgcccttctt |
| | growth factor | | (Sequence No. 27) | (Sequence No. 28) |
| AY029180.1 | plasminogen activator, | PLAUR | acaccaccaaatgcaacga | ccccttgcagctgtaacac |
| | urokinase receptor | | (Sequence No. 29) | (Sequence No. 30) |
| NM_005988.2 | small proline-rich | SPRR2A | aacccctggtacctgagca | cttgcactgctgctgttgat |
| | protein 2A | | (Sequence No. 31) | (Sequence No. 32) |
| AK293280.1 | semaphorin 7A, GPI | SEMA7A | cctttcatgtgctttacctaactaca | gatgttgaaggcgaagctgt |
| | membrane anchor (John Milton | | (Sequence No. 33) | (Sequence No. 34) |
| | Hagen blood group) | | | |
| NM_033503.3 | Bcl2 modifying factor | BMF | agttccaccggcttcatgt | tcttctccattcaaagcaagg |
| | | | (Sequence No. 35) | (Sequence No. 36) |
| NM_003106.2 | SRY (sex determining | SOX2 | tgctgcctctttaagactaggac | cctggggctcaaacttctct |
| | region Y)-box 2 | | (Sequence No. 37) | (Sequence No. 38) |
| M31842.1 | hypoxanthine-phosphoribosyl- | HPRT1 | tgaccttgatttattttgcatacc | cgagcaagacgttcagtcct |
| | tranferase 1 | | (Sequence No. 39) | (Sequence No. 40) |

TABLE 5
A verification result using qPCR.

| | | Fold change | | | |
|---|---|---|---|---|---|
| | | (qPCR) | | Fold change | |
| Representative | Gene | 0.5 ug/ | | (cDNA microarray) | |
| Public ID | Symbol | ml | 1 ug/ml | 0.5 ug/ml | 1 ug/ml |
| AK226147.1 | SPRY4 | 4.9 | 8.9 | 8.901 | 7.916 |
| BC073983.1 | EGR1 | 7.5 | 12.8 | 8.263 | 7.355 |
| BT007067.1 | IL8 | 9.4 | 10.2 | 7.348 | 8.188 |
| AK222688.1 | HBEGF | 9.3 | 10.4 | 5.996 | 5.896 |
| NM_001136178.1 | EGR2 | 9.4 | 6.8 | 5.887 | 5.999 |
| CX756248 | RHOB | 4 | 5.4 | 5.234 | 4.993 |
| NM_016270.2 | KLF2 | 5.5 | 5.2 | 4.551 | 4.210 |
| NM_000758.2 | CSF2 | 12.7 | 12 | 4.138 | 4.167 |
| NM_006290.2 | TNFAIP3 | 4.4 | 5.9 | 4.060 | 4.000 |
| BC005047.1 | DUSP6 | 4.7 | 4.9 | 3.676 | 3.520 |
| AI446767 | CXCL2 | 4.5 | 5 | 3.438 | 3.343 |
| NM_013246.2 | CLCF1 | 3.8 | 5.1 | 3.152 | 2.984 |
| NM_013376.3 | SERTAD1 | 3.3 | 3.6 | 3.123 | 3.161 |
| NM_001901.2 | CTGF | 5.4 | 5.3 | 3.041 | 3.368 |
| AY029180.1 | PLAUR | 2.6 | 2.4 | 2.564 | 2.716 |
| NM_005988.2 | SPRR2A | 4 | 4.4 | 1.947 | 2.175 |
| AK293280.1 | SEMA7A | 2.3 | 2.4 | 1.816 | 2.327 |
| NM_033503.3 | BMF | 0.33 | 0.56 | 0.346 | 0.317 |
| NM_003106.2 | SOX2 | 0.68 | 0.49 | 0.207 | 0.211 |

### Example 3

### Verification of Selected Genes

3-1. Evaluation of Anti-c-Met Antibodies Using Selected Genes from NCI-H441 Cells

To verify whether genes selected using a microarray may be used in functional evaluation of anti-c-Met antibodies, qPCR was performed for four types of genes representing different pathways. EGR1 is known to be important in cell growth, HBEGF has an apoptosis suppression function, and CSF2 is known to play an important role in inflammation. A qPCR was processed by using NCI-H441 cells with respect to four different genes. As a negative control group, a mouse IgG was used and as a positive control group, a well-known agent 5D5 was used. Antibodies used in evaluation were anti-c-Met antibodies that are variants of L3-1Y.

Antibodies were diluted to a concentration of 1 μg/mL in RPMI1640 without FBS for two hours.

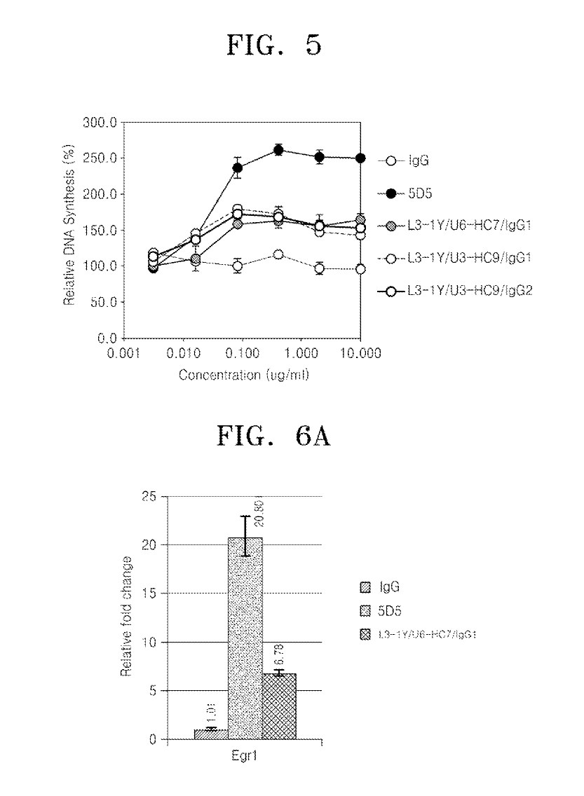

As shown in Table 3, the L3-1Y variant induces substantially lower expression compared to 5D5 and this is similar to a result of measuring side effects using a BrdU assay of FIG. 5.

3-2. Verification of Genes Selected by Using Caki-1 Cells

To verify whether genes identified through a microarray using NCI-H441 cells are applicable in other cell lines, similar experiments were performed using Caki-1 cells (renal cancer cell line). First, to extract RNA, Caki-1 cells (ATCC) were divided into 6 well plates, each having a concentration of 2.0×10⁵ cells/well and cultured for two days. A mouse IgG used as a negative control group, a well-known agent 5D5, and L3-1Y TH7 were diluted to a concentration of 1 μg/mL in RPMI1640 (GIBCO) without FBS and treated for two hours. Processes of RNA preparation and qPCR are the same as NCI-H441 cells.

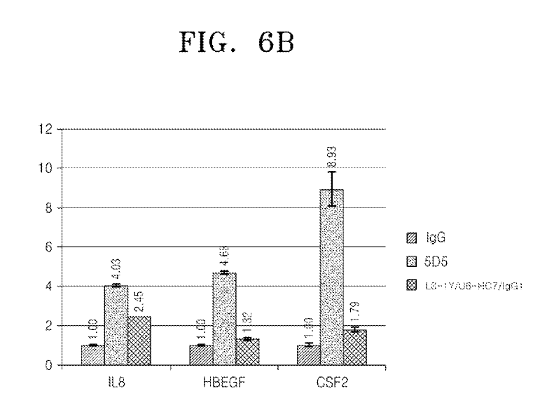

As shown in FIGS. 6A and 6B, treatment with anti-c-Met antibodies having great side effects (5D5), gene expression increased and treatment with antibodies having relatively small side effects (L3-1Y variants), gene expression was less than that of 5D5. This result is similar to a result of NCI-H441 and showed that selected genes are applicable to other cell lines.

What is claimed is:

1. A DNA microarray chip comprising one or more oligonucleotide sequences selected from the group consisting of:
SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2 (GenBank Accession No.: NM_{—}000758.2), TNFAIP3 (GenBank Accession No.: NM_{—}006290.2), KDM6B (GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1 (GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8 (GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), G0S2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F(GenBank Accession No.: NM_{—}001014450.1), ITPRIP(GenBank Accession No.: NM_{—}033397.2), DCUN1D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), SOCS3 (GenBank Accession No.: NM_{—}003955.3), BMP2 (GenBank Accession No.: M22489.1), ZNF451 (GenBank Accession No.: BC021712.2), LOC643008 (GenBank Accession No.: AK055768.1), CDKN1B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2(GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2), or portion thereof or sequence complementary thereto.

2. A method of screening anti-c-Met antibody having reduced side effects, the method comprising:
(1) treating a cancer cell with a test compound to provide an experimental group;
(2) isolating RNA from the experimental group treated with the sample compound, and from a control group cancer cell that is not treated with the test compound;
(3) producing cDNA from the RNA of the experimental group and the control group, and marking the cDNA of the experimental group and the control group with different fluorescent markers;
(4) hybridizing the cDNA of each of the experimental and control groups to a DNA microarray chip of claim 1 to determine gene expression of the experimental and control groups; and
(5) comparing the gene expression of the experimental and control groups.

3. The method of claim 2, wherein the cancer cell is a NCI-H441 cell or Caki-1 cell.

4. The method of claim 2, wherein the fluorescent markers are selected from the group consisting of Cy3, Cy5, poly L-lysine-fluorescein isothiocyanate (FITC), rhodamine-B-isothiocyanate (RITC), and rhodamine.

5. The method of claim 2, wherein enhanced expression of one or more of the following genes in the experimental group as compared to the control group identifies an anti-c-Met antibody having reduced side effects:
SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2(GenBank Accession No.: NM_{—}000758.2), TNFAIP3(GenBank Accession No.: NM_{—}006290.2), KDM6B(GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1 (GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8 (GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), GOS2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F (GenBank Accession No.: NM_{—}001014450.1), ITPRIP (GenBank Accession No.: NM_{—}033397.2), DCUN1D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1 RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), and SOCS3 (GenBank Accession No.: NM_{—}003955.3), BMP2 (GenBank Accession No.: M22489.1).

6. The method of claim 2, wherein reduced expression of one or more of the following genes in the experimental group as compared to the control group identifies an anti-c-Met antibody having reduced side effects:
ZNF451 (GenBank Accession No.: BC021712.2), LOC643008 (GenBank Accession No.: AK055768.1), CDKN1 B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2 (GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2).

7. A method of identifying c-Met-antibodies having reduced side effects, the method comprising:
(1) treating a cancer cell with a test compound to provide an experimental group;
(2) isolating RNA from the experimental group treated with the test compound and from a control group cancer cell that is not treated with the test compound;
(3) performing real-time reverse transcript polymerase chain reaction (RT-PCR) with the RNA from the experimental group and the control group using a primer complementary to a biomarker gene and capable of amplifying the biomarker gene to produce a biomarker gene product; and
(4) comparing expression of the biomarker gene products of step 3) of the experimental group and the control group
wherein the biomarker gene is one or more selected from the group consisting of SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2 (GenBank Accession No.: NM_{—}000758.2), TNFAIP3 (GenBank Accession No.: NM_{—}006290.2), KDM6B (GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1 (GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8 (GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), GOS2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F(GenBank Accession No.: NM_{—}001014450.1), ITPRIP(GenBank Accession No.: NM_{—}033397.2), DCUN1D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), SOCS3 (GenBank Accession No.: NM_{—}003955.3), BMP2 (GenBank Accession No.: M22489.1), ZNF451 (GenBank Accession No.: BC021712.2), LOC643008 (GenBank Accession No.: AK055768.1), CDKN1B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2(GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2).

8. The method of claim 7, wherein the cancer cell is NCI-H441 cell or Caki-1 cell.

9. The method of claim 7, wherein at least two forward-direction or reverse-direction primers are used, which primers are selected from the group consisting of SEQ ID NOs: 1 to 40.

10. The method of claim 7, wherein enhanced expression of one or more of the following biomarker genes in the experimental group as compared to the control group identifies an anti-c-Met antibody having reduced side effects:
SPRY4 (GenBank Accession No.: AK226147.1), EGR1 (GenBank Accession No.: BC073983.1), IL8 (GenBank Accession No.: BT007067.1), HBEGF (GenBank Accession No.: AK222688.1), EGR2 (GenBank Accession No.: NM_{—}001136178.1), RHOB (GenBank Accession No.: CX756248), KLF2 (GenBank Accession No.: NM_{—}016270.2), PLK3 (GenBank Accession No.: CR607047.1), SPRR2D (GenBank Accession No.: NM_{—}006945.4), CSF2(GenBank Accession No.: NM_{—}000758.2), TNFAIP3(GenBank Accession No.: NM_{—}006290.2), KDM6B(GenBank Accession No.: BU685696), GDF15 (GenBank Accession No.: NM_{—}004864.2), DUSP6 (GenBank Accession No.: BC005047.1), SPRR2E (GenBank Accession No.: NM_{—}001024209.2), DUSP5 (GenBank Accession No.: NM_{—}004419.3), CXCL2 (GenBank Accession No.: AI446767), FOS (GenBank Accession No.: AK298659.1), OVOL1 (GenBank Accession No.: NM_{—}004561.2), MAFF (GenBank Accession No.: NM_{—}012323.2), CLCF1 (GenBank Accession No.: NM_{—}013246.2), ZNF697 (GenBank Accession No.: BM904784), SERTAD1 (GenBank Accession No.: NM_{—}013376.3), LIF (GenBank Accession No.: AI570795), CTGF (GenBank Accession No.: NM_{—}001901.2), EGR4 (GenBank Accession No.: NM_{—}001965.2), TMEM88 (GenBank Accession No.: NM_{—}203411.1), CSRNP1 (GenBank Accession No.: BM129310), ZFP36L1 (GenBank Accession No.: BC018340.1), EPHA2 (GenBank Accession No.: NM_{—}004431.2), PTGER4 (GenBank Accession No.: NM_{—}000958.2), FOSB (GenBank Accession No.: NM_{—}006732.2), ARID3B (GenBank Accession No.: AK298716.1), ANGPTL4 (GenBank Accession No.: NM_{—}139314.1), PHLDA2 (GenBank Accession No.: CB991991), C17orf91 (GenBank Accession No.: NM_{—}001001870.1), BCL2A1 (GenBank Accession No.: NM_{—}001114735.1), CYR61 (GenBank Accession No.: AK295430.1), LOC730755 (GenBank Accession No.: BC063625.1), MFSD2A (GenBank Accession No.: AF370364.1), NR4A1 (GenBank Accession No.: NM_{—}002135.3), PLAUR (GenBank Accession No.: AY029180.1), KRT17 (GenBank Accession No.: g197383031), CDKN1A (GenBank Accession No.: NM_{—}078467.1), CXCL3 (GenBank Accession No.: NM_{—}002090.2), CLN8 (GenBank Accession No.: AF123760.1), SPRR1B (GenBank Accession No.: NM_{—}003125.2), ADM (GenBank Accession No.: BF589790), JUNB (GenBank Accession No.: NM_{—}002229.2), KRT16///KRT16P1///KRT16P2///KRT16P3 (GenBank Accession No.: AK301679.1), GEM (GenBank Accession No.: NM_{—}005261.2), BHLHE40 (GenBank Accession No.: BC082238.1), NR4A3 (GenBank Accession No.: D78579.1), ACSL4 (GenBank Accession No.: AK294197.1), ITPKC (GenBank Accession No.: NM_{—}025194.2), AEN (GenBank Accession No.: AK022624.1), TRAF1 (GenBank Accession No.: BC024145.2), PFKFB2 (GenBank Accession No.: AB044805.1), NIPAL1 (GenBank Accession No.: NM_{—}207330.1), TRIB1 (GenBank Accession No.: NM_{—}025195.2), KBTBD8 (GenBank Accession No.: NM_{—}032505.2), DIDO1 (GenBank Accession No.: BX097024), ZYX (GenBank Accession No.: NM_{—}003461.4), KRT16P1 (GenBank Accession No.: AK301679.1), PHLDA1 (GenBank Accession No.: BC110820.1), KCTD11 (GenBank Accession No.: NM_{—}001002914.2), IER3 (GenBank Accession No.: NM_{—}003897.3), GOS2 (GenBank Accession No.: BE874862), FOSL1 (GenBank Accession No.: NM_{—}005438.3), CD274 (GenBank Accession No.: AK300470.1), DUSP2 (GenBank Accession No.: NM_{—}004418.3), MCL1 (GenBank Accession No.: BC107735.1), ARC (GenBank Accession No.: NM_{—}015193.3), ATF3 (GenBank Accession No.: NM_{—}001040619.1), JUN (GenBank Accession No.: BG491844), MFNG (GenBank Accession No.: NM_{—}002405.2), CCRN4L (GenBank Accession No.: BC023512.2), EREG (GenBank Accession No.: BC136404.1), CXCL1 (GenBank Accession No.: NM_{—}001511.1), TP53RK (GenBank Accession No.: AB065434.1), METRNL (GenBank Accession No.: BC118558.1), SERTAD2 (GenBank Accession No.: BC101639.1), FLJ36031 (GenBank Accession No.: BC013906.2), PTX3 (GenBank Accession No.: BC039733.1), DOT1L (GenBank Accession No.: AF509504.1), CCL20 (GenBank Accession No.: NM_{—}004591.2), CD83 (GenBank Accession No.: AK290426.1), AMOTL2 (GenBank Accession No.: CN364627), JMJD6 (GenBank Accession No.: CR602714.1), ZBTB24 (GenBank Accession No.: BC036731.1), MCPH1 (GenBank Accession No.: BC030702.1), HNRNPC (GenBank Accession No.: AK302213.1), NCOA7 (GenBank Accession No.: BQ003857), SOCS4 (GenBank Accession No.: AF424815.1), DUSP1 (GenBank Accession No.: AK299391.1), CDC42EP2 (GenBank Accession No.: AF098290.1), YRDC (GenBank Accession No.: NM_{—}024640.3), PRSS22 (GenBank Accession No.: BX356243), MARS2 (GenBank Accession No.: NM_{—}138395.2), SPRR2A (GenBank Accession No.: NM_{—}005988.2), SLC25A25 (GenBank Accession No.: NM_{—}052901.2), PTGS2 (GenBank Accession No.: BC013734.1), TICAM1 (GenBank Accession No.: NM_{—}182919.2), RPS27A (GenBank Accession No.: BC042362.1), SPRR2F (GenBank Accession No.: NM_{—}001014450.1), ITPRIP (GenBank Accession No.: NM_{—}033397.2), DCUN1D3 (GenBank Accession No.: NM_{—}173475.2), SPOCD1 (GenBank Accession No.: NM_{—}144569.4), SAMD4A (GenBank Accession No.: AF429970.1), RRAD (GenBank Accession No.: NM_{—}001128850.1), C6orf141 (GenBank Accession No.: NM_{—}001145652.1), LAMB3 (GenBank Accession No.: AK296851.1), IL1 RL1 (GenBank Accession No.: AK303389.1), SEMA7A (GenBank Accession No.: AK293280.1), ZNF562 (GenBank Accession No.: AK304370.1), C8orf4 (GenBank Accession No.: AA702805), MAP3K14 (GenBank Accession No.: NM_{—}003954.2), and SOCS3 (GenBank Accession No.: NM_{—}003955.3), BMP2 (GenBank Accession No.: M22489.1).

11. The method of claim 7, wherein reduced expression of one or more of the following biomarker genes in the experimental group as compared to the control group identifies an anti-c-Met antibody having reduced side effects:
ZNF451 (GenBank Accession No.: BC021712.2), LOC643008 (GenBank Accession No.: AK055768.1), CDKN1 B (GenBank Accession No.: NM_{—}004064.3), ALPP (GenBank Accession No.: NM_{—}001632.3), NHLRC1 (GenBank Accession No.: NM_{—}198586.2), ZNF624 (GenBank Accession No.: NM_{—}020787.2), KBTBD7 (GenBank Accession No.: AK299614.1), LOC100506379 (GenBank Accession No.: CR622974.1), LIPT2 (GenBank Accession No.: NM_{—}001144869.1), BMF (GenBank Accession No.: NM_{—}033503.3), ZBED2 (GenBank Accession No.: NM_{—}024508.3), and SOX2 (GenBank Accession No.: NM_{—}003106.2).

12. A kit for screening anti-c-Met antibodies having reduced side effects comprising the DNA microarray chip of claim 1.


SEQUENCE LISTING (42 sequences)

SEQ ID NO: 1 - DNA, 18 bp, Artificial Sequence
  Synthetic (Sense primer of SPRY4)
ccccggcttcaggattta

SEQ ID NO: 2 - DNA, 20 bp, Artificial Sequence
  Synthetic (antisense primer of SPRY4)
ctgcaaaccgctcaatacag

SEQ ID NO: 3 - DNA, 19 bp, Artificial Sequence
  Synthetic (antisense primer of EGR1)
tcggacatgacagcaacct

SEQ ID NO: 4 - DNA, 20 bp, Artificial Sequence
  Synthetic (antisense primer of EGR1)
tttcccctttccctttagca

SEQ ID NO: 5 - DNA, 20 bp, Artificial Sequence
  Synthetic (Sense primer of IL8)
agacagcagagcacacaagc

SEQ ID NO: 6 - DNA, 18 bp, Artificial Sequence
  Synthetic (antisense primer of IL8)
atggttccttccggtggt

SEQ ID NO: 7 - DNA, 20 bp, Artificial Sequence
  Synthetic (Sense primer of HBEGF)
tggggcttctcatgtttagg

SEQ ID NO: 8 - DNA, 21 bp, Artificial Sequence
  Synthetic (antisense primer of HBEGF)
catgcccaacttcactttctc

SEQ ID NO: 9 - DNA, 20 bp, Artificial Sequence
  Synthetic (Sense primer of EGR2)
ttgaccagatgaacggagtg

SEQ ID NO: 10 - DNA, 21 bp, Artificial Sequence
  Synthetic (antisense primer of EGR2)
tggtttctaggtgcagagacg

SEQ ID NO: 11 - DNA, 19 bp, Artificial Sequence
  Synthetic (Sense primer of RHOB)
tatgtggccgacattgagg

SEQ ID NO: 12 - DNA, 18 bp, Artificial Sequence
  Synthetic (antisense primer of RHOB)
gcggtcgtagtcctcctg

SEQ ID NO: 13 - DNA, 18 bp, Artificial Sequence
  Synthetic (Sense primer of KLF2)
catctgaaggcgcatctg

SEQ ID NO: 14 - DNA, 19 bp, Artificial Sequence
  Synthetic (antisense primer of KLF2)
cgtgtgctttcggtagtgg

SEQ ID NO: 15 - DNA, 22 bp, Artificial Sequence
  Synthetic (Sense primer of CSF2)
tctcagaaatgtttgacctcca

SEQ ID NO: 16 - DNA, 18 bp, Artificial Sequence
  Synthetic (antisense primer of CSF2)
gcccttgagcttggtgag

SEQ ID NO: 17 - DNA, 21 bp, Artificial Sequence
  Synthetic (Sense primer of TNFAIP3)
tgcacactgtgtttcatcgag

SEQ ID NO: 18 - DNA, 20 bp, Artificial Sequence
  Synthetic (antisense primer of TNFAIP3)
acgctgtgggactgactttc

SEQ ID NO: 19 - DNA, 20 bp, Artificial Sequence
  Synthetic (Sense primer of DUSP6)
cgactggaacgagaatacgg

SEQ ID NO: 20 - DNA, 20 bp, Artificial Sequence
  Synthetic (antisense primer of DUSP6)
ggagaactcggcttggaact

SEQ ID NO: 21 - DNA, 22 bp, Artificial Sequence
  Synthetic (Sense primer of CXCL2)
cccatggttaagaaaatcatcg

SEQ ID NO: 22 - DNA, 20 bp, Artificial Sequence
  Synthetic (antisense primer of CXCL2)
cttcaggaacagccaccaat

SEQ ID NO: 23 - DNA, 22 bp, Artificial Sequence
  Synthetic (Sense primer of CLCF1)
ccagaaaacctatgacctcacc

SEQ ID NO: 24 - DNA, 20 bp, Artificial Sequence
  Synthetic (antisense primer of CLCF1)
gggcccaggtagttcagata

SEQ ID NO: 25 - DNA, 19 bp, Artificial Sequence
  Synthetic (Sense primer of SERTAD1)
tctgattggccgctagtga

SEQ ID NO: 26 - DNA, 19 bp, Artificial Sequence
  Synthetic (antisense primer of SERTAD1)
cgactgccagaggttcctt

SEQ ID NO: 27 - DNA, 20 bp, Artificial Sequence
  Synthetic (Sense primer of CTGF)
ctcctgcaggctagagaagc

SEQ ID NO: 28 - DNA, 21 bp, Artificial Sequence
  Synthetic (antisense primer of CTGF)
gatgcactttttgcccttctt

SEQ ID NO: 29 - DNA, 19 bp, Artificial Sequence
  Synthetic (Sense primer of PLAUR)
acaccaccaaatgcaacga

SEQ ID NO: 30 - DNA, 19 bp, Artificial Sequence
  Synthetic (antisense primer of PLAUR)
ccccttgcagctgtaacac

SEQ ID NO: 31 - DNA, 19 bp, Artificial Sequence
  Synthetic (Sense primer of SPRR2A)
aacccctggtacctgagca

SEQ ID NO: 32 - DNA, 20 bp, Artificial Sequence
  Synthetic (antisense primer of SPRR2A)
cttgcactgctgctgttgat

SEQ ID NO: 33 - DNA, 26 bp, Artificial Sequence
  Synthetic (Sense primer of SEMA7A)
cctttcatgtgctttacctaactaca

SEQ ID NO: 34 - DNA, 20 bp, Artificial Sequence
  Synthetic (antisense primer of SEMA7A)
gatgttgaaggcgaagctgt

SEQ ID NO: 35 - DNA, 19 bp, Artificial Sequence
  Synthetic (Sense primer of BMF)
agttccaccggcttcatgt

SEQ ID NO: 36 - DNA, 21 bp, Artificial Sequence
  Synthetic (antisense primer of BMF)
tcttctccattcaaagcaagg

SEQ ID NO: 37 - DNA, 23 bp, Artificial Sequence
  Synthetic (Sense primer of SOX2)
tgctgcctctttaagactaggac

SEQ ID NO: 38 - DNA, 20 bp, Artificial Sequence
  Synthetic (antisense primer of SOX2)
cctggggctcaaacttctct

SEQ ID NO: 39 - DNA, 24 bp, Artificial Sequence
  Synthetic (Sense primer of HPRT1)
tgaccttgatttattttgcatacc

SEQ ID NO: 40 - DNA, 20 bp, Artificial Sequence
  Synthetic (antisense primer of HPRT1)
cgagcaagacgttcagtcct

SEQ ID NO: 41 - protein, 462 aa, Artificial Sequence
  Synthetic (polypeptide consisting of heavy chain variable region of huAbF46-H4-A1, U6-HC7 hinge and constant region of human IgG1)
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1410 bp, Artificial Sequence
  Synthetic (polynucleotide encoding polypeptide consisting of heavy chain variable region of huAbF46-H4-A1, U6-HC7 hinge and constant region of human IgG1)
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag